(19) United States
(12) Patent Application Publication
(10) Pub. No.: US 2026/0158194 A1
(43) Pub. Date: Jun. 11, 2026

(54) DRUG COATED BALLOON

(71) Applicant: ARTERIUS LIMITED, Leeds (GB)

(72) Inventors: Kadem AL-LAMEE, Leeds (GB); Nial BULLETT, Leeds (GB); Lydia SMITH, Leeds (GB); Naveed AHMED, Leeds (GB); William GROVE, Leeds (GB)

(21) Appl. No.: 18/706,487

(22) PCT Filed: Nov. 3, 2022
(86) PCT No.: PCT/GB2022/052777

§ 371 Date: May 1, 2024

(30) Foreign Application Priority Data
Nov. 3, 2021 (GB) 2115813.4

(51) Int. Cl. A61L 29/16; A61K 31/436; A61K 31/519; A61L 29/08; A61M 25/10

(52) U.S. Cl. CPC A61L 29/16; A61K 31/436; A61K 31/519; A61L 29/085; A61M 2025/1031; A61M 2025/105

(57) ABSTRACT

A coating composition for application to an expandable medical device including a polymeric compound including a central portion and two end portions wherein either the central portion is hydrophobic and the end portions are hydrophilic or the central portion is hydrophilic and the end portions are hydrophobic; at least one active agent wherein at least one active agent is hydrophobic when the central portion of the polymeric compound is hydrophobic and at least one active agent is hydrophilic when the central portion of the polymeric compound is hydrophilic; and a solvent; when in use the at least one active agent is encapsulated by the polymeric compound; and at least one active agent is a vasodilator and/or an anti-platelet agent. The invention extends to a coating composition system comprising the coating composition.

### FIELD

The present invention relates to a coating composition and coating system for an expandable medical device comprising a polymeric compound and at least one active agent. The present invention extends to an expandable medical device coated with a composition derived from said coating composition as well as a method of coating an expandable medical device.

### BACKGROUND

Coronary Artery Disease (CAD) is the foremost single cause of mortality and loss of disability adjusted life years globally. This accounts for nearly 7 million deaths annually (J Epidemiology Glob Health. 2021).

Peripheral arterial disease (PAD) is estimated to affect over 200 million people around the world and this number is increasing continuously. Endovascular interventions such as percutaneous transluminal angioplasty (PTA) with or without stenting are among the preferred choices for the treatment of PAD.

Recent randomised controlled clinical trials have demonstrated the superiority of drug coated balloon (DCB) therapy when compared to PTA alone, in terms of improved patency and reduced target lesion revascularisation. In many clinical cases, DCB devices offer an alternative to drug-eluting stents (DES), while also avoiding the need for a permanent implant, allowing shorter medications and avoiding additional/multiple stent layers.

In most of the commercially available DCB products, an anti-cancer drug, paclitaxel, has been the drug of choice. The main challenge in the effectiveness of this approach is the efficient delivery of drug to the target site. For current DCBs, as much as 90% of drug is lost in the blood stream, with only between 1-10% successfully delivered (Cardiovascular Research Technologies, 2013). The effects of a drug or other component of the formulation lost downstream are potentially harmful, but paclitaxel in particular inhibits proliferation of any cell type in any organ. Having a consistent and safe drug dose that is stable on introduction into the body, provides a precise percentage transfer into the vessel wall and achieves a long-term pharmacokinetic profile to prevent restenosis is desirable.

### DESCRIPTION OF EMBODIMENTS

It is an object of aspects of the present invention to provide one or more solutions to the above-mentioned problems.

According to a first aspect of the invention there is provided a coating composition for application to an expandable medical device comprising a polymeric compound comprising a central portion and two end portions wherein either the central portion is hydrophobic and the end portions are hydrophilic or the central portion is hydrophilic and the end portions are hydrophobic; at least one active agent wherein the active agent is hydrophobic when the central portion of the polymeric compound is hydrophobic and the active agent is hydrophilic when the central portion of the polymeric compound is hydrophilic; and a solvent; wherein in use the at least one active agent is encapsulated by the polymeric compound; and wherein at least one active agent is a vasodilator and/or an anti-platelet agent.

According to a second aspect of the invention there is provided a coating composition for application to an expandable medical device comprising a polymeric compound comprising a central portion and two end portions wherein either the central portion is hydrophobic and the end portions are hydrophilic or the central portion is hydrophilic and the end portions are hydrophobic; a plurality of active agents wherein at least one active agent is hydrophobic when the central portion of the polymeric compound is hydrophobic and at least one active agent is hydrophilic when the central portion of the polymeric compound is hydrophilic; and a solvent; wherein in use the at least one active agent is encapsulated by the polymeric compound.

Preferably, at least one active agent is hydrophobic and/or hydrophilic.

Preferably, at least one active agent is a vasodilator.

According to a third aspect of the present invention there is provided a coating system for application to an expandable medical device, the or each coating system comprising:
a) a first coating composition comprising a first polymeric compound comprising a central portion and two end portions wherein either the central portion is hydrophobic and the end portions are hydrophilic or the central portion is hydrophilic and the end portions are hydrophobic; a first active agent wherein the first active agent is hydrophobic when the central portion of the first polymeric compound is hydrophobic and the first active agent is hydrophilic when the central portion of the first polymeric compound is hydrophilic; and a first solvent; wherein in use the first active agent is encapsulated by the first polymeric compound; and wherein the first active agent is a vasodilator and/or an anti-platelet agent; and
b) a second coating composition comprising a second polymeric compound comprising a central portion and two end portions wherein either the central portion is hydrophobic and the end portions are hydrophilic or the central portion is hydrophilic and the end portions are hydrophobic; a second active agent wherein the second active agent is hydrophobic when the central portion of the second polymeric compound is hydrophobic and the second active agent is hydrophilic when the central portion of the second polymeric compound is hydrophilic; and a second solvent; wherein in use the second active agent is encapsulated by the second polymeric compound;
and wherein the first and second active agents are different.

By encapsulated is intended to mean that in use the active agent is confined by the polymeric compound.

By having both hydrophobic and hydrophilic properties the polymeric compound is capable of encapsulating the active agent(s) for release at the desired time. It also maximises adhesion of the coating to the surface of the expandable medical device and transfer of the active agents to the point of treatment.

The polymeric compound may be any suitable polymeric compound. Preferably the hydrophobic portion is selected from the group comprising fatty acid derivatives, polybutylene oxide and polypropylene oxide, preferably the hydrophobic portion is polypropylene oxide. Preferably the hydrophilic portion is selected from the group comprising ethylene oxide derivatives, preferably the hydrophilic portion is polyethylene glycol. Preferably the polymeric compound is a triblock copolymer, preferably a poloxamer derivative. Poloxamers, also known by the trade names Pluronic® and Synperonic™, are non-ionic triblock copolymers.

Many different poloxamers exist that have slightly different properties. For the generic term poloxamer, these copolymers are commonly named with the letter P (for poloxamer) followed by three digits: the first two digits multiplied by 100 give the approximate molecular mass of the polyoxypropylene core, and the last digit multiplied by 10 gives the percentage polyoxyethylene content (e.g. P407=poloxamer with a polyoxypropylene molecular mass of 4000 g/mol} and a 70% polyoxyethylene content). For the Pluronic® and Synperonic™ tradenames, coding of these copolymers starts with a letter to define its physical form at room temperature (L=liquid, P=paste, F=flake (solid)) followed by two or three digits. The first digit (or first two digits in a three-digit number) in the numerical designation, multiplied by 300, indicates the approximate molecular weight of the hydrophobe; and the last digit multiplied by 10 gives the percentage polyoxyethylene content (e.g., L61 indicates a polyoxypropylene molecular mass of 1800 g/mol and a 10% polyoxyethylene content).

Typically the polymeric compound is selected from one or more of poly(ethylene glycol)-block-poly(propylene glycol)-block-poly(ethylene glycol) diacrylate, poly(ethylene glycol)-block-poly(propylene glycol)-block-poly(ethylene glycol) dimethacrylate, O,O′-bis(2-aminopropyl) polypropylene glycol-block-polyethylene glycol-block-polypropylene glycol, polyethylene glycol-polypropylene oxide-polyethylene glycol [PEG-PPO-PEG], and polypropylene oxide-polyethylene glycol-polypropylene-polypropylene oxide [PPO-PEG-PPO]. The structures of PEG-PPO-PEG and PPO-PEG-PPO are shown below:

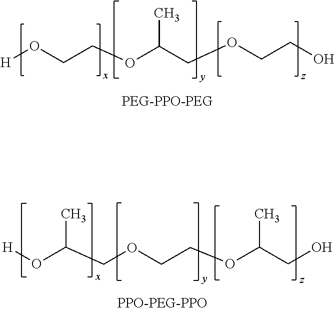

Preferably the polymeric compound is PEG-PPO-PEG or PPO-PEG-PPO, most preferably PEG-PPO-PEG.

Preferably the Mₙ of the polymeric compound is at least 2,500 Da, such as at least 5,000 Da, such as at least 10,000 Da, such as at least 12,500 Da, such as at least 13,000 Da. Most preferably the Mₙ of the polymeric compound is at least 10,000 Da, such as at least 12,500 Da, such as at least 13,000 Da. Preferably the Mn of the polymeric compound is from 2,500 to 50,000 Da, more preferably from 5,000 to 30,000 Da, preferably from 10,000 and 20,000 Da, more preferably from 12,500 to 20,000 Da, most preferably from 13,000 to 20,000 Da. The Mₙ of the polymeric compound may be from 12,500 to 50,000 Da, such as from 13,000 to 50,000 Da. It has been found that by using a polymeric compound of the preferred Mₙ, a coating formed from the coating composition has better properties for usability such as lower tack.

In a preferred embodiment, the polymeric compound may have an Mₙ: of approximately 14,600 Da.

The molecular weight (Mₙ) of the polymeric compound may be at least 14,000 Da. Having a molecular weight of at least 14,000 Da may increase the adhesion to the surface of the expandable medical device. Having a molecular weight of at least 14,000 Da may mean that the polymer, in use, can encapsulate a high number of active agent molecules.

Alternatively, the molecular weight of the polymeric compound may be <14,000 Da. Having a molecular weight of <14,000 Da may ensure both the polymeric compound and the organic solvent where used are both in the liquid phase. The or each coating composition may be a homogeneous solution.

Typically, the end portions of the polymeric compound will have a Mₙ of 350-16,000 Da, typically approximately 12,000 Da. Typically, the central portion of the polymeric compound will have a Mₙ of 900-5,000 Da, typically approximately 2,900 Da. Typically, the end portions of the polymeric compound comprise between 2 and 1150 repeat units, preferably between 5 and 800 repeat units or 50 and 350 repeat units, typically approximately 275 repeat units. Typically, the central portions of the polymeric compound comprise between 1 and 850 repeat units, preferably between 5 and 500 repeat units or between 10 and 100 repeat units, typically approximately 50 repeat units.

The or each coating composition may comprise a plurality of active agents. The or each coating composition may comprise two, three, four, five, etc. active agents. Preferably, the or each coating composition may comprise two active agents. Preferably, the or each coating composition may comprise two active agents that have different effects, such as one active agent that is an anti-proliferative agent and one active agent that is a vasodilator and/or anti-platelet agent.

The active agent(s) may be hydrophobic and/or hydrophilic. A person skilled in the art would understand that an active agent may be hydrophobic, may be hydrophilic or may have both hydrophobic and hydrophilic portions. When an active agent has both hydrophobic and hydrophilic portions, the active agent may be more hydrophobic than hydrophilic or may be more hydrophilic than hydrophobic. By “more hydrophobic than hydrophilic” is meant that the active agent typically acts as a hydrophobic molecule (in the context of the invention). For the avoidance of doubt an active agent that is more hydrophobic than hydrophilic may be construed as a hydrophobic active agent herein. By “more hydrophilic than hydrophobic” is meant that the active agent typically acts as a hydrophilic molecule (in the context of the invention). For the avoidance of doubt an active agent that is more hydrophilic than hydrophobic may be construed as a hydrophilic active agent herein.

Preferably, the or each coating composition may comprise at least one active agent that is more hydrophilic than hydrophobic.

The or each coating composition may comprise at least one hydrophobic active agent, i.e., at least one active agent is hydrophobic. Typically, small molecule active agents have hydrophobic properties.

The or each coating composition may comprise at least one hydrophilic active agent, i.e., at least one active agent may be hydrophilic.

The active agents may comprise at least one hydrophobic active agent and at least one hydrophilic active agent, such as a hydrophobic active agent and a hydrophilic active agent. For example, when the or each coating composition comprises a plurality of active agents, the or each coating composition may comprise at least one hydrophobic active agent, i.e., at least one active agent is hydrophobic, and at least one hydrophilic active agent, i.e., at least one active agent may be hydrophilic. As such, the or each coating composition may comprise at least one hydrophobic active agent and at least one hydrophilic active agent, such as a hydrophobic active agent and a hydrophilic active agent.

The active agents may comprise a plurality of hydrophobic agents. As such, the or each coating composition may comprise a plurality of hydrophobic agents.

Preferably, the active agents may comprise one hydrophobic agent and one hydrophilic agent. As such, preferably, the or each coating composition may comprise one hydrophobic agent and one hydrophilic agent.

In preferred embodiments the or each coating composition comprises an anti-proliferative agent and/or a vasodilator and/or anti-platelet agent. In particularly preferred embodiments the or each coating composition comprises a plurality of active agents. In preferred embodiments the or each coating composition comprises an anti-proliferative agent and a vasodilator and/or an anti-platelet agent.

The anti-proliferative agent may be selected from one of 17 beta-estadiol, alpha-interferon, angiopeptin, argatroban, aspirin, azathioprine, Biolimus A9, bivalirudin, captopril, chloromethylketone, cilazapril, clobetasol, colchicines, dexamethasone, dextran, dipyridamole, docetaxel doxorubicin hydrochloride, ethylrapamycin, everolimus, fluorouracil, forskolin, genistein, heparin, a low molecular weight heparin, heparinoid, hirudin, recombinant hirudin, lovastatin, methotrexate, mitomycin, nifedipine, nitroprusside, paclitaxel, permirolast pimecrolimus, potassium, prostacyclin, prostacyclin analogue, rapamycin (sirolimus), serotonin blocker suramin, sodium heparin, super oxide dismutase mimetic, tacrolimus, temsirolimus, thioprotease inhibitor, triazolopyrimidine, vapiprost, vinblastine, vincristine, zotarolimus, and any salts or analogues thereof.

The vasodilator and/or anti-platelet agent may be one of, but is not limited to one of alprostadil, amlodipine, benazepril, bencyclane, captopril, clevidipine, clopidogrel, cyclandelate, Diazoxide, diltiazem, dipyridamole, enalapril, ergoloid, fasudil, felodipine, fenoldopam, fosinopril, hydralazine, hydroergocristine, ifenprodil, inositol, isosorbide dinitrate, isosorbide mononitrate, isradipine, lacidipine, levamlodipine, lisinopril, mesylate, miacin, minoxidil, moexipril, moxisylyte, nesiritide, nicardipine, nicergoline, nicotinate, nifedipine, nimodipine, nisoldipine, nitrogylcerin, nitroprusside, nylidrin, nitric oxide, perindopril, phenoxybenzamine, phentolamine, pentifylline, pentoxifylline quinapril, ramipril, riociguat, theophylline and derivatives, tolazoline, trandolapril, verapamil, vericiguat, xanthinol, and any salts or analogues thereof.

A person skilled in the art would know which of the above-mentioned anti-proliferative agents and vasodilator and/or anti-platelet agents have hydrophobic portions and which have hydrophilic portions.

Interaction between the anti-proliferative agent and the vasodilator and/or anti-platelet agent should be complimentary or should exhibit no adverse interactions which inhibit efficacy or enhance effects of the active agent.

Preferably the anti-proliferative agent is sirolimus. A person skilled in the art would know that sirolimus is hydrophobic.

Preferably the vasodilator and/or anti-platelet agent is dipyridamole. A person skilled in the art would know that dipyridamole is more hydrophilic than hydrophobic (and, as such, acts as a hydrophilic active agent in the context of the present invention).

Preferably the anti-proliferative agent is sirolimus and the vasodilator and/or anti-platelet agent is dipyridamole. Advantageously, when dipyridamole is used in combination with sirolimus, the dipyridamole, which is a vasodilator, may enhance the amount of sirolimus that is released in a blood vessel.

The or each coating composition solution comprises a solvent. The solvent is typically an organic solvent. The organic solvent may be selected from methanol, acetonitrile, dichloromethane, hexane, toluene, acetone, ethanol, chloroform, tetrachloroethylene, acetic acid, tetrahydrofuran, 1-butanol, 2-butanol, ethyl acetate, formamide, triethylamine, cyclohexane, dimethyl ether, dioxan, and benzene. Preferably the solvent is selected from the group comprising acetonitrile, chloroform, dichloromethane, ethanol, ethyl acetate and methanol or mixtures thereof. Preferably the solvent is methanol.

The organic solvent is typically compatible to dissolve both the polymeric compound and the one or more active agents and should not chemically attack the material of the expandable medical device.

The active agents may each typically be present in the organic solvent at less than 10% by weight, preferably less than 5% by weight, preferably less than 2% by weight, more preferably less than 1% by weight. By using a lower percent by weight of active agent, the coated expandable medical devices typically are more durable.

The polymeric compound may typically be present in the organic solvent at less than or equal to 50% by weight, preferably less than or equal to 40%, less than or equal to 30%, less than or equal to 20%, less than or equal to 10%, less than or equal to 5%, or even less than or equal to 2% by weight.

The or each coating composition may be a dip coating composition. By “dip coating composition” is meant that the coating composition is operable to be applied to an expandable medical device by a dip coating method.

Preferably, when the or each coating composition is a dip coating composition the polymeric compound is present in the organic solvent at less than or equal to 50% by weight, preferably less than or equal to 40%, less than or equal to 30%, or less than or equal to 25% by weight. Preferably, when the or each coating composition is a dip coating composition the polymeric compound is present in the organic solvent from 1% to 50% by weight, preferably from 1 to 30% by weight, preferably from 5% to 30% by weight, preferably from 10% to 30% by weight, preferably from 15% to 25% by weight, most preferably approximately 20% by weight.

Preferably, when the or each coating composition is a dip coating composition each active agent may typically be present in the organic solvent at less than 30% by weight, preferably less than 20% by weight, preferably less than 15% by weight, most preferably less than 10% by weight. Preferably, when the or each coating composition is a dip coating composition each active agent may typically be present in the organic solvent from 1% to 30% by weight, preferably from 1% to 20% by weight, preferably from 5% to 20% by weight, preferably from 5% to 15% by weight, most preferably from 5% to 10% by weight.

The or each coating composition may be a spray coating composition. By “spray coating composition” is meant that the coating composition is operable to be applied to an expandable medical device by a spray coating method.

Preferably, when the or each coating composition is a spray coating composition the polymeric compound is present in the organic solvent at less than or equal to 20%, less than or equal to 10%, less than or equal to 5%, less than or equal to 2%, or even less than or equal to 1.5% by weight. Preferably, when the or each coating is a spray coating composition the polymeric compound is present in the organic solvent from 0.5% to 20% by weight, preferably from 0.5 to 15% by weight, preferably from 0.5% to 10% by weight, more preferably from 0.5% to 5% by weight, more preferably from 0.5% to 2% by weight, more preferably from 1% to 2% by weight most preferably approximately 1% by weight.

Preferably, when the or each coating composition is a spray coating composition each active agent may typically be present in the organic solvent at less than 30% by weight, preferably less than 20% by weight, preferably less than 15% by weight, preferably less than 10% by weight, preferably less than 5% by weight, most preferably less than 2.5% by weight. Preferably, when the or each coating composition is a spray coating composition each active agent may typically be present in the organic solvent from 0.5% to 30% by weight, preferably from 0.5% to 20% by weight, preferably from 1% to 10% by weight, preferably from 1% to 5% by weight, preferably from 1% to 2.5%, most preferably approximately 2% by weight.

The invention extends to a coating system for application to an expandable medical device, the coating system comprising:
a) a first coating composition comprising a first polymeric compound comprising a central portion and two end portions wherein either the central portion is hydrophobic and the end portions are hydrophilic or the central portion is hydrophilic and the end portions are hydrophobic; a first active agent wherein the first active agent is hydrophobic when the central portion of the first polymeric compound is hydrophobic and the first active agent is hydrophilic when the central portion of the first polymeric compound is hydrophilic; and a first solvent; wherein in use the first active agent is encapsulated by the first polymeric compound; and wherein the first active agent is a vasodilator and/or an anti-platelet agent; and
b) a second coating composition comprising a second polymeric compound comprising a central portion and two end portions wherein either the central portion is hydrophobic and the end portions are hydrophilic or the central portion is hydrophilic and the end portions are hydrophobic; a second active agent wherein the second active agent is hydrophobic when the central portion of the second polymeric compound is hydrophobic and the second active agent is hydrophilic when the central portion of the second polymeric compound is hydrophilic; and a second solvent; wherein in use the second active agent is encapsulated by the polymeric compound;
and wherein the first and second active agents are different.

The first coating composition may be applied to an expandable medical device before or after the second coating composition. Further coating layers may also be applied. For example, a further coating layer may be applied before the first and second coating compositions. For example, a further coating layer may be applied after the first and second coating compositions. For example, a further coating layer may be applied between the first and second coating compositions.

Preferably the first active agent is dipyridamole.

Preferably the second active agent is sirolimus.

Preferably the first active agent is dipyridamole and the second active agent is sirolimus.

The first and second polymeric compound may be the same or may be different. Suitable polymeric compounds are as defined herein. Preferably the first and second polymeric compounds are the same.

The first and second solvent may be the same or may be different. Suitable solvents are as defined herein. Preferably the first and second solvents are the same.

The coating compositions and/or coating systems are suitable for application to the surface of an expandable medical device. Hence according to a fourth aspect of the invention there is provided an expandable medical device coated on at least a portion of the surface thereof with a coating, the coating being derived from a coating composition according to the first and/or second aspects of the invention and/or a coating system according to the third aspect of the present invention. The or each coating derived from said coating composition(s) will typically be substantially free of solvent.

According to a fifth aspect of the invention, there is provided a method of delivering an active agent or a plurality of active agents to a target site in a lumen of a human or animal body for treating a condition associated with said lumen comprising: locating an expandable medical device according to the fourth aspect of the invention at the target site; and expanding the expandable medical device such that the surface of the device is in contact with the target site.

The expandable medical device of the fourth or fifth aspect is preferably a device suitable for delivering an active agent or a plurality of active agents to a target site in a lumen of a human or animal body. The lumen may be a blood vessel, a duct, a urethra or a ureter, preferably a blood vessel.

Typically, the device is a balloon catheter. The balloon catheter may be used for percutaneous transluminal angioplasty (PTA), or percutaneous transluminal coronary angioplasty (PTCA) and other drug delivery catheter treatments. This balloon catheter typically comprises a catheter with an inflatable balloon mounted on the catheter. Suitable balloon catheters will be known to those skilled in the art.

The balloon catheter may be selected to be of a relevant size for the size of vessel desired to be treated. The inflatable balloon surface on the catheter may be made from any suitable material such as a derivative of nylon, polyethylene terephthalate or a polyether block amide such as Pebax®.

The inflatable portion of the balloon has a hydrophobic or hydrophilic surface, which interacts with the relevant hydrophobic or hydrophilic portion of the polymeric compound. This enables the polymeric compound to form a good adhesion to the hydrophobic or hydrophilic surface to form a durable coating. Typically, the balloon catheter has a hydrophobic surface.

According to a further aspect of the invention is also provided a method of coating an expandable medical device comprising the steps of:
(i) providing a coating composition according to the first and/or second aspect of the invention;
(ii) applying the coating composition of step (i) to at least a portion of the surface of the expandable medical device; and optionally;
(iii) applying a further coating composition comprising a polymeric compound comprising a central portion and two end portions wherein either the central portion is hydrophobic and the end portions are hydrophilic, or the central portion is hydrophilic and the end portions are hydrophobic and a solvent to at least a portion of the surface of the expandable medical device.

Step (iii) may be performed before or after step (ii). Preferably after step (ii). Without wishing to be bound by theory, by performing step (iii) after step (ii) the percentage of drug released at the desired site is typically higher. When more than one active agent is present, the active agents and polymeric compound may be combined before the coating composition is applied to allow the release of both active agents to be at a similar rate.

Without being bound by theory, it is speculated that the release rate of each active agent may be different in-vivo, for example the release rate of one active agent may be higher than another.

Step (ii) may be repeated to apply a greater amount of the active agent to the surface of the expandable medical device. Step (ii) may be repeated as many times as is necessary, typically at least twice, such as 3, 4, 5, 6, 7, 8, 9, 10 or more times.

Step (ii) may further include the step of removing the solvent in the coating composition.

Said removal may be active or passive, i.e., the solvent may be allowed to evaporate or may be actively removed by any suitable method, such as heating, or applying a vacuum etc.

Step (ii) may be repeated with a different active agent present in the coating composition according to the first aspect of the invention in each repeated step (ii). Without wishing to be bound by theory it is believed that in doing so the adhesion of each active agent and its polymeric compound to the surface of the expandable medical device is increased. The release rate of each active agent may also be controlled by this method.

Thus, the invention extends to a coating system comprising a first and second coating composition, wherein each coating composition comprises a polymeric compound comprising a central portion and two end portions wherein either the central portion is hydrophobic and the end portions are hydrophilic, or the central portion is hydrophilic and the end portions are hydrophobic, at least one active agent, wherein the active agent is hydrophobic when the central portion of the polymeric compound is hydrophobic and the active agent is hydrophilic when the central portion of the polymeric compound is hydrophilic, and a solvent, and wherein the first and second coating compositions comprise a different active agent.

The active agent of the first or second coating composition may comprise a vasodilator and/or anti-proliferative agent.

Thus, according to a further aspect of the invention is also provided a method of coating an expandable medical device comprising the steps of:
(i) providing a coating system according to the third aspect of the invention; and
(ii) applying the coating system of step (i) to at least a portion of the surface of the expandable medical device; and optionally
(iii) applying a further coating composition comprising a third polymeric compound comprising a central portion and two end portions wherein either the central portion is hydrophobic and the end portions are hydrophilic, or the central portion is hydrophilic and the end portions are hydrophobic and a third solvent to at least a portion of the surface of the expandable medical device.

Step (iii) may be performed before or after step (ii). Step (ii) may be performed during step (ii). For example, the further coating composition may be applied between the first and second coating compositions of the coating composition system.

Step (ii) may be repeated to apply a greater amount of the active agent to the surface of the expandable medical device. Step (ii) may be repeated as many times as is necessary, typically at least twice, such as 3, 4, 5, 6, 7, 8, 9, 10 or more times. Each coating composition of the coating composition system may individually be applied at least once, such as 2, 3, 4, 5, 6, 7, 8, 9, 10 or more times.

Step (ii) may further include the step of removing the solvent in each coating composition of the coating composition system. The solvent may be removed from each coating of the coating composition system at the same time or at different times. Preferably the solvent is removed from one coating composition, suitably to form a coating, before a further coating composition is applied. Said removal may be active or passive, i.e., the solvent may be allowed to evaporate or may be actively removed by any suitable method, such as heating, or applying a vacuum etc.

The coating composition and/or coating system may be applied to the surface of the expandable medical device by any suitable method. Suitable methods include dip coating, spray coating, brushing, spinning or inkjet printing. Typically, the or each coating composition may be applied by dip coating or spray coating.

Dip coating may increase adhesion of the coating the or each coating to the surface of the expandable medical device.

Spray coating may ensure a more precise dosage of the active agent may be applied to the surface of the expandable medical device.

As used herein, ‘by weight’ in relation to percent by weight of a component in solvent, means the ratio of weight/volume (expressed as a percentage) wherein the weight is the weight of the component in grams (g), and wherein the volume is the volume of the solvent in mL.

The invention is further defined in the following numbered aspects:
1. A coating composition for application to an expandable medical device comprising a polymeric compound comprising a central portion and two end portions wherein either the central portion is hydrophobic and the end portions are hydrophilic or the central portion is hydrophilic and the end portions are hydrophobic; a plurality of active agents wherein at least one active agent is hydrophobic when the central portion of the polymeric compound is hydrophobic and at least one active agent is hydrophilic when the central portion of the polymeric compound is hydrophilic; and a solvent; wherein in use the at least one active agent is encapsulated by the polymeric compound.
2. The coating composition of aspect 1 wherein an active agent is dipyridamole.
3. The coating composition of any preceding aspect wherein an active agent is sirolimus.
4. The coating composition any preceding aspect wherein the active agents comprise an anti-proliferative agent and a vasodilator and/or anti-platelet agent.
5. The coating composition of aspect 4 wherein the anti-proliferative agent is sirolimus and the vasodilator and/or anti-platelet agent is dipyridamole.
6. The coating composition of any preceding aspect wherein at least one active agent is hydrophobic and/or hydrophilic, for example hydrophobic, or for example hydrophilic.
7. The coating composition of any preceding aspect wherein the polymeric compound is a triblock copolymer.
8. The coating composition of any preceding aspect wherein the Mn of the polymeric compound is at least 10,000 Da, preferably at least 12,500 Da, more preferably at least 13,000 Da, more preferably from 12,500 to 50,000 Da, such as from 13,000 to 50,000 Da.
9. The coating composition of any preceding aspect wherein the triblock copolymer is a poloxamer derivative.
10. The coating composition of any preceding aspect wherein the coating composition is a dip coating composition; and wherein each active agent is present in the organic solvent at less than 30% by weight, preferably less than 20% by weight, preferably less than 15% by weight, most preferably less than 10% by weight.
11. The coating composition of any aspect 10 wherein the coating composition is a dip coating composition; and wherein each active agent is present in the organic solvent from 1% to 30% by weight, preferably from 1% to 20% by weight, preferably from 5% to 20% by weight, preferably from 5% to 15% by weight, most preferably from 5% to 10% by weight.
12. The coating composition of any one of aspects 1 to 9 wherein the coating composition is a spray coating composition; and wherein each active agent is present in the organic solvent at less than 30% by weight, preferably less than 20% by weight, preferably less than 15% by weight, preferably less than 10% by weight, preferably less than 5% by weight, most preferably less than 2.5% by weight.
13. The coating composition of aspect 12 wherein the coating composition is a spray coating composition; and wherein each active agent is present in the organic solvent from 0.5% to 30% by weight, preferably from 0.5% to 20% by weight, preferably from 1% to 10% by weight, preferably from 1% to 5% by weight, preferably from 1% to 2.5%, most preferably approximately 2% by weight.
14. The coating composition of any preceding aspect wherein the polymeric compound is present in the solvent at less than 50% by weight, preferably less than or equal to 40%, less than or equal to 30%, or less than or equal to 25% by weight.
15. The coating composition of any one of aspects 1 to 9, 10, 11 or 14 wherein the coating composition is a dip coating composition; and wherein the polymeric compound is present in the organic solvent at less than or equal to 50% by weight, preferably less than or equal to 40%, less than or equal to 30%, or less than or equal to 25% by weight.
16. The coating composition of aspect 15 wherein the coating composition is a dip coating composition; and wherein the polymeric compound is present from 1% to 50% by weight, preferably from 1 to 30% by weight, preferably from 5% to 30% by weight, preferably from 10% to 30% by weight, preferably from 15% to 25% by weight, most preferably approximately 20% by weight.
17. The coating composition of any one of aspects 1 to 9, 12, 13, or 14 wherein the coating composition is a spray coating composition; and wherein the polymeric compound is present in the solvent at less than or equal to 20%, less than or equal to 10%, less than or equal to 5%, less than or equal to 2%, or even less than or equal to 1.5% by weight.
18. The coating composition of aspect 17, wherein the coating composition is a spray coating composition; and wherein the polymeric compound is present from 0.5% to 20% by weight, preferably from 0.5 to 15% by weight, preferably from 0.5% to 10% by weight, more preferably from 0.5% to 5% by weight, more preferably from 0.5% to 2% by weight, more preferably from 1% to 2% by weight most preferably approximately 1% by weight.
19. A coating composition for application to an expandable medical device comprising a polymeric compound comprising a central portion and two end portions wherein either the central portion is hydrophobic and the end portions are hydrophilic or the central portion is hydrophilic and the end portions are hydrophobic; at least one active agent wherein the active agent is hydrophobic when the central portion of the polymeric compound is hydrophobic and the active agent is hydrophilic when the central portion of the polymeric compound is hydrophilic; and a solvent; wherein in use the at least one active agent is encapsulated by the polymeric compound; and wherein at least one active agent is a vasodilator and/or an anti-platelet agent.
20. The coating composition of aspect 19 wherein the at least one active agent is hydrophobic and/or hydrophilic, for example hydrophobic, or for example hydrophilic.
21. The coating composition of aspect 19 or 20 wherein the vasodilator and/or an anti-platelet agent is dipyridamole.
22. The coating composition of any one of aspects 19 to 21 wherein the coating composition comprises a plurality of active agents.
23. The coating composition of any one of aspects 19 to 22 wherein an active agent is sirolimus.
24. The coating composition of any one of aspects 19 to 23 wherein the active agents comprise an anti-proliferative agent and a vasodilator and/or anti-platelet agent.
25. The coating composition of aspect 24 wherein the anti-proliferative agent is sirolimus and the vasodilator and/or anti-platelet agent is dipyridamole.
26. The coating composition of any one of aspects 19 to 24 wherein at least one active agent is hydrophobic and/or hydrophilic, for example hydrophobic, or for example hydrophilic.
27. The coating composition of any one of aspects 19 to 26 wherein the polymeric compound is a triblock copolymer.
28. The coating composition of any one of aspects 19 to 27 wherein the Mn of the polymeric compound is at least 10,000 Da, preferably at least 12,500 Da, more preferably at least 13,000 Da, more preferably from 12,500 to 50,000 Da, such as from 13,000 to 50,000 Da.
29. The coating composition of any one of aspects 19 to 28 wherein the triblock copolymer is a poloxamer derivative.
30. The coating composition of any one of aspects 19 to 29 wherein the coating composition is a dip coating composition; and wherein each active agent is present in the organic solvent at less than 30% by weight, preferably less than 20% by weight, preferably less than 15% by weight, most preferably less than 10% by weight.
31. The coating composition of aspect 30 wherein the coating composition is a dip coating composition; and wherein each active agent is present in the organic solvent from 1% to 30% by weight, preferably from 1% to 20% by weight, preferably from 5% to 20% by weight, preferably from 5% to 15% by weight, most preferably from 5% to 10% by weight.
32. The coating composition of one of aspects 19 to 29 wherein the coating composition is a spray coating composition; and wherein each active agent is present in the organic solvent at less than 30% by weight, preferably less than 20% by weight, preferably less than 15% by weight, preferably less than 10% by weight, preferably less than 5% by weight, most preferably less than 2.5% by weight.
33. The coating composition of aspect 32 wherein the coating composition is a spray coating composition; and wherein each active agent is present in the organic solvent from 0.5% to 30% by weight, preferably from 0.5% to 20% by weight, preferably from 1% to 10% by weight, preferably from 1% to 5% by weight, preferably from 1% to 2.5%, most preferably approximately 2% by weight.
34. The coating composition of any one of aspects 19 to 33 wherein the polymeric compound is present in the solvent at less than 50% by weight, preferably less than or equal to 40%, less than or equal to 30%, or less than or equal to 25% by weight.
35. The coating composition of any one of aspects 19 to 29, 30, 31 or 34 wherein the coating composition is a dip coating composition; and wherein the polymeric compound is present in the organic solvent at less than or equal to 50% by weight, preferably less than or equal to 40%, less than or equal to 30%, or less than or equal to 25% by weight.
36. The coating composition of aspect 35 wherein the coating composition is a dip coating composition; and wherein the polymeric compound is present from 1% to 50% by weight, preferably from 1 to 30% by weight, preferably from 5% to 30% by weight, preferably from 10% to 30% by weight, preferably from 15% to 25% by weight, most preferably approximately 20% by weight.
37. The coating composition of any one of aspects 19 to 29, 32, 33 or 34 wherein the coating composition is a spray coating composition; and wherein the polymeric compound is present in the solvent at less than or equal to 20%, less than or equal to 10%, less than or equal to 5%, less than or equal to 2%, or even less than or equal to 1.5% by weight.
38. The coating composition of aspect 37, wherein the coating composition is a spray coating composition; and wherein the polymeric compound is present from 0.5% to 20% by weight, preferably from 0.5 to 15% by weight, preferably from 0.5% to 10% by weight, more preferably from 0.5% to 5% by weight, more preferably from 0.5% to 2% by weight, more preferably from 1% to 2% by weight most preferably approximately 1% by weight.
39. A coating composition system for application to an expandable medical device, the coating system comprising:
a) a first coating composition comprising a first polymeric compound comprising a central portion and two end portions wherein either the central portion is hydrophobic and the end portions are hydrophilic or the central portion is hydrophilic and the end portions are hydrophobic; a first active agent wherein the first active agent is hydrophobic when the central portion of the first polymeric compound is hydrophobic and the first active agent is hydrophilic when the central portion of the first polymeric compound is hydrophilic; and a first solvent; wherein in use the first active agent is encapsulated by the first polymeric compound; and wherein the first active agent is a vasodilator and/or an anti-platelet agent; and
b) a second coating composition comprising a second polymeric compound comprising a central portion and two end portions wherein either the central portion is hydrophobic and the end portions are hydrophilic or the central portion is hydrophilic and the end portions are hydrophobic; a second active agent wherein the second active agent is hydrophobic when the central portion of the second polymeric compound is hydrophobic and the second active agent is hydrophilic when the central portion of the second polymeric compound is hydrophilic; and a second solvent; wherein in use the second active agent is encapsulated by the polymeric compound;
and wherein the first and second active agents are different.
40. The coating composition system of aspect 39 wherein the first and second active agents are independently hydrophilic and/or hydrophobic, for example wherein the first active agent is hydrophilic and the second active agent is hydrophobic.
41. The coating composition system of aspect 39 or 40 wherein first active agent is dipyridamole and/or the second active agent is sirolimus.
42. The coating composition system of any one of aspects 39 to 41 wherein the first and/or second coating composition comprises a plurality of active agents.
43. The coating composition system of any one of aspects 39 to 42 wherein the second active agent is an anti-proliferative agent.
44. The coating composition system of any one of aspects 39 to 43 wherein the first and/or second polymeric compound is a triblock copolymer, for example wherein each of the first and second polymeric compound is a triblock copolymer.
45. The coating composition system of any one of aspects 39 to 44 wherein the Mn of the first and/or second polymeric compound is at least 10,000 Da, preferably at least 12,500 Da, more preferably at least 13,000 Da, more preferably from 12,500 to 50,000 Da, such as from 13,000 to 50,000 Da, for example wherein the Mn of each of the first and second polymeric compound is at least 10,000 Da, preferably at least 12,500 Da, more preferably at least 13,000 Da, more preferably from 12,500 to 50,000 Da, such as from 13,000 to 50,000 Da.
46. The coating composition of any one of aspects 39 to 45 wherein the triblock copolymer is a poloxamer derivative.
47. The coating composition of any one of aspects 39 to 46 wherein the first and/or second coating composition is a dip coating composition; and wherein each active agent is present in the organic solvent at less than 30% by weight, preferably less than 20% by weight, preferably less than 15% by weight, most preferably less than 10% by weight, for example wherein the first and second coating composition are each a dip coating composition; and wherein each active agent is present in the organic solvent at less than 30% by weight, preferably less than 20% by weight, preferably less than 15% by weight, most preferably less than 10% by weight.
48. The coating composition of aspect 47 wherein the first and/or second coating composition is a dip coating composition; and wherein each active agent is present in the organic solvent from 1% to 30% by weight, preferably from 1% to 20% by weight, preferably from 5% to 20% by weight, preferably from 5% to 15% by weight, most preferably from 5% to 10% by weight, for example wherein the first and second coating composition are each a dip coating composition; and wherein each active agent is present in the organic solvent from 1% to 30% by weight, preferably from 1% to 20% by weight, preferably from 5% to 20% by weight, preferably from 5% to 15% by weight, most preferably from 5% to 10% by weight.
49. The coating composition of one of aspects 39 to 46 wherein the first and/or second coating composition is a spray coating composition; and wherein each active agent is present in the organic solvent at less than 30% by weight, preferably less than 20% by weight, preferably less than 15% by weight, preferably less than 10% by weight, preferably less than 5% by weight, most preferably less than 2.5% by weight, for example wherein the first and second coating composition are each a spray coating composition; and wherein each active agent is present in the organic solvent at less than 30% by weight, preferably less than 20% by weight, preferably less than 15% by weight, preferably less than 10% by weight, preferably less than 5% by weight, most preferably less than 2.5% by weight.
50. The coating composition of aspect 49 wherein the first and/or second coating composition is a spray coating composition; and wherein each active agent is present in the organic solvent from 0.5% to 30% by weight, preferably from 0.5% to 20% by weight, preferably from 1% to 10% by weight, preferably from 1% to 5% by weight, preferably from 1% to 2.5%, most preferably approximately 2% by weight, for example wherein the first and second coating composition are each a spray coating composition; and wherein each active agent is present in the organic solvent from 0.5% to 30% by weight, preferably from 0.5% to 20% by weight, preferably from 1% to 10% by weight, preferably from 1% to 5% by weight, preferably from 1% to 2.5%, most preferably approximately 2% by weight.
51. The coating composition of any one of aspects 28 to 36 wherein the first and/or second polymeric compound is present in the solvent at less than 50% by weight, preferably less than or equal to 40%, less than or equal to 30%, or less than or equal to 25% by weight, for example wherein the first and second polymeric compound are each present in the solvent at less than 50% by weight, preferably less than or equal to 40%, less than or equal to 30%, or less than or equal to 25% by weight.
52. The coating composition of any one of aspect 39 to 46, 47, 48 or 51 wherein the first and/or second coating composition is a dip coating composition; and wherein the polymeric compound is present in the organic solvent at less than or equal to 50% by weight, preferably less than or equal to 40%, less than or equal to 30%, or less than or equal to 25% by weight, for example wherein the first and second coating composition are each a dip coating composition; and wherein the polymeric compound is present in the organic solvent at less than or equal to 50% by weight, preferably less than or equal to 40%, less than or equal to 30%, or less than or equal to 25% by weight.
53. The or each coating composition of aspect 52 wherein the first and/or second coating composition is a dip coating composition; and wherein the polymeric compound is present from 1% to 50% by weight, preferably from 1 to 30% by weight, preferably from 5% to 30% by weight, preferably from 10% to 30% by weight, preferably from 15% to 25% by weight, most preferably approximately 20% by weight, for example wherein the first and second coating composition are each a dip coating composition; and wherein the polymeric compound is present from 1% to 50% by weight, preferably from 1 to 30% by weight, preferably from 5% to 30% by weight, preferably from 10% to 30% by weight, preferably from 15% to 25% by weight, most preferably approximately 20% by weight.
54. The coating composition of any one of aspects 39 to 46, 49, 50 or 51 wherein the first and/or second coating composition is a spray coating composition; and wherein the polymeric compound is present in the solvent at less than or equal to 20%, less than or equal to 10%, less than or equal to 5%, less than or equal to 2%, or even less than or equal to 1.5% by weight, for example wherein the first and second coating composition are each a spray coating composition; and wherein the polymeric compound is present in the solvent at less than or equal to 20%, less than or equal to 10%, less than or equal to 5%, less than or equal to 2%, or even less than or equal to 1.5% by weight.
55. The coating composition of aspect 54, wherein the first and/or second coating composition is a spray coating composition; and wherein the polymeric compound is present from 0.5% to 20% by weight, preferably from 0.5 to 15% by weight, preferably from 0.5% to 10% by weight, more preferably from 0.5% to 5% by weight, more preferably from 0.5% to 2% by weight, more preferably from 1% to 2% by weight most preferably approximately 1% by weight, for example wherein the first and second coating composition are each a spray coating composition; and wherein the polymeric compound is present from 0.5% to 20% by weight, preferably from 0.5 to 15% by weight, preferably from 0.5% to 10% by weight, more preferably from 0.5% to 5% by weight, more preferably from 0.5% to 2% by weight, more preferably from 1% to 2% by weight most preferably approximately 1% by weight.
56. An expandable medical device coated on at least a portion of the surface thereof with a coating, the coating being derived from a coating composition according to any one of aspects 1 to 38, and/or a coating system, the coating system being derived from a coating composition system according to any one of aspects 39 to 55.
57. A method of coating an expandable medical device comprising the steps of:
(i) providing a coating composition according to any one of aspects 1 to 38 and/or a coating composition system according to any one of aspects 39 to 55;
(ii) applying the coating composition and/or coating composition system of step (i) to at least a portion of the surface of the expandable medical device; and optionally
(iii) applying a mixture of a polymeric compound comprising a central portion and two end portions wherein either the central portion is hydrophobic and the end portions are hydrophilic or the central portion is hydrophilic and the end portions are hydrophobic and optionally a solvent to at least a portion of the surface of the expandable medical device coated in step (ii).
58. A method according to aspect 57 wherein the coating composition and/or coating composition system is applied by dip coating or spray coating.
59. A method of delivering at least one active agent, such as a plurality of active agents, to a target site in a lumen of a human or animal body for treating a condition associated with said lumen comprising: locating an expandable medical device according to aspect 56 at the target site; and expanding the expandable medical device such that the surface of the device is in contact with the target site.
60. The coating composition of any one of aspects 1 to 38, coating composition system of any one of aspects 39 to 55, expandable medical device of aspect 56 or the method of any one of aspects 57 to 59 wherein the device is a balloon catheter.
61. The coating composition, coating composition system, expandable medical device or method of aspect 60 wherein the balloon catheter has a hydrophobic or hydrophilic surface.
62. The coating composition, coating composition system, expandable medical device or method of aspect 61, wherein the balloon catheter has a hydrophobic surface.

All of the features contained herein may be combined with any of the above aspects in any combination.

For a better understanding of the invention, and to show how embodiments of the same may be carried into effect, reference will now be made, by way of example, to the following examples.

### EXAMPLES

### Example 1

A single layer of coating was applied to a balloon surface using a 1%, single-drug methanol solution with varying ratios of excipient polymers. This was achieved using dip coating. Four solutions were used—a 50% PEG-PPO-PEG solution, a 5% PEG-PPO-PEG solution, a 50% PPO-PEG-PPO solution and a 5% PPO-PEG-PPO solution. Each coating solution was dip-coated on to four individual balloons followed by overnight drying at room temperature.
PEG-PPO-PEG Mₙ value: 2,900
PPO-PEG-PPO Mₙ value: 2,700

Coated balloons were then eluted in phosphate buffered saline (PBS) to discern the retention of the coating integrity on the balloon surface.

The evaluations made showed that balloons coated with the 5% solutions had an improved coating integrity over the balloons coated with the 50% solutions, after eluting in PBS.

### Example 2

PEG-PPO-PEG of varying molecular weights was assessed for coating uniformity and adhesion on the balloon surface. A single layer of coating was applied to a balloon surface using a 1%, single-drug methanol solution with varying molecular weight polymers followed by overnight drying at room temperature. This was achieved using dip coating. Two solutions were used—both being a 5% PEG-PPO-PEG type with the following molecular weights:
PEG-PPO-PEG Mₙ value: 2,900—Viscous liquid
PEG-PPO-PEG Mₙ value: 14,600—White powder

Balloons coated with the higher molecular weight polymer rendered a more uniform, durable and dry coating, whereas coating with lower molecular weight polymer resulted in a non-uniform and tacky coating which easily delaminated from the balloon surface.

Multiple layers may be applied by dip coating. Using 5% polymer, one or three dip-coating cycles were performed on the balloon, using the 14,600 Mw PEG-PPO-PEG polymer in powder form, dissolved in methanol.

### Example 3

For this example, a single drug formulation was used, which was dipyridamole, present as 1% of the solution. The coatings were applied to the balloons as single- and triple-coatings.

Following drying of the coatings, balloons were dipped in 8 mL PBS for three minutes to assess the stability of the coating and loss of drug. Visual inspection of the balloon and coating was performed, and drug content in the PBS was evaluated by UV-VIS spectrophotometry.

Presence and quantity of dipyridamole was detected in the PBS by absorbance at the 415 nm characteristic wavelength of the drug by UV VIS, as shown in Table 1.

TABLE 1

| PBS Blank | Absorbance at 415 nm |
|---|---|
| 1 Coat | 0.068 |
| 3 Coats | 0.077 |

After three-minute elution in PBS, the release of the drug was similar for the single- and triple-coated balloons.

Methanol release of the remaining coating was performed to quantify the total drug content in the single- and triple-coated balloons.

Visual inspection of the balloon surfaces after two-minute elution in methanol showed complete loss of all coating from the balloon surface.

Presence and quantity of dipyridamole was detected in the methanol by absorbance at the 415 nm characteristic wavelength of the drug by UV VIS, as shown in Table 2.

TABLE 2

| Methanol Blank | Absorbance at 415 nm |
|---|---|
| 1 Coat | 0.099 |
| 3 Coats | 0.297 |

Total content of drug in the coated surfaces was calculated by summing the PBS and methanol released quantities.

While a similar quantity of drug was released in PBS for both the single- and triple-coated balloons, the triple-coated balloon showed 3 times the total drug content, suggesting that there is more total drug available in the coating which will be able to transfer to the vessel wall.

### Example 4

The technique of spray coating was introduced using a 5% solution of high molecular weight polymer PEG-PPO-PEG (Mₙ=14,600) in methanol. This spray coating method was used to add multiple layers of coating and ensured the correct area of the balloon was coated in a more precise fashion.

In this example, a set of experiments was conducted to assess the drug release profiles of Sirolimus and Dipyridamole. The samples were spray coated with their coating solutions with nitrogen gas at 1 bar, and 20 rotations were done (10 clockwise and 10 anticlockwise). The separation from the balloon and spray nozzle was approximately 15 cm.

The drug release profiles of both Dipyridamole and Sirolimus were determined using an excipient polymer PEG-PPO-PEG (Mₙ=14,600) with organic carrier solvent methanol. The coating solution quantities can be found in Table 3.

TABLE 3

| | |
|---|---|
| Dipyridamole | Sirolimus |

| | | | | |
|---|---|---|---|---|
| Polymer | 0.5105 | g | 0.5123 | g |
| Drug | 0.1039 | g | 0.0964 | g |
| Methanol | 10 | mL | 10 | mL |

The resulting coated balloons were eluted in 8 mL of PBS for 3 minutes, before being eluted into 8 mL of methanol for 5 minutes, for full release of the coating.

### Dipyridamole

Post-PBS reading @415 nm: 0.042
Post-Methanol reading @415 nm: 0.087

### Sirolimus

Post-PBS reading @279 nm: 0.103
Post-Methanol reading @279 nm: 0.166

From the UV reading and a calibration coefficient, it was calculated that during the 5-minute elution time 32.6% Dipyridamole was released and 38.3% Sirolimus was released, therefore meaning that Sirolimus has a slightly faster rate of release than that of Dipyridamole from the PEG-PPO-PEG polymer.

### Example 5

In this example, a set of experiments was conducted to assess what percentage of polymer was most effective at retaining a drug on the surface of the balloons (using a microscope only). Each coated balloon was dipped in 8 mL of PBS for three minutes to evaluate the coating surface.

Four balloons were coated with differing quantities of polymer (from Example 2) and dipyridamole as below:
A: 1% Polymer=>0.3039 g polymer in 30 mL methanol
B: 2% Polymer=>0.6239 g polymer in 30 mL methanol
C: 1% Polymer & 1% Dipyridamole solution=>0.3714 g polymer & 0.3265 g drug in 30 mL methanol
D: 2% Polymer & 1% Dipyridamole solution=>0.6620 g polymer & 0.324 g drug in 30 mL methanol Balloons were spray coated using the following parameters: Nitrogen gas rate: 0.5 bar, 20 rotations of the balloon (10 clockwise, 10 anticlockwise) at a rate of 1 rotation per second. The separation from the balloon and spray nozzle was approximately 15 cm. Each coated balloon was then dipped in PBS for 3 minutes. Area visual inspection of each balloon showed that for Balloon A: the polymer was still visible on the surface; Balloon B: the polymer was still visible on the surface; Balloon C: the drug was still very visible on the surface; and Balloon D: the drug was still visible on the surface.

After 3 mins eluting in PBS, some of the drug coating is lost; however, the majority of the coating remained in each case.

### Example 6

In this example, several different layered coating systems were compared to determine their drug release profiles and coating integrity. Balloons were spray coated using the following parameters: Nitrogen gas rate: 0.5 bar, 20 rotations of the balloon (10 clockwise, 10 anticlockwise) at a rate of 1 rotation per second. The separation from the balloon and spray nozzle was approximately 15 cm. The drying time between layers was 1.5 hrs. The balloons were pleated and folded, tracked through an anatomical model, then held in PBS for 3 minutes at 37° C. where it was inflated. This PBS dip represents the elution at the desired site. The balloons were then deflated, retracted and dipped in 8 mL of methanol to release the remaining coating and drug.

Table 4 shows the percentage of drug released at the desired site for each coating configuration.

TABLE 4

| | | | Absorbance | Absorbance | |
|---|---|---|---|---|---|
| Sample | Layer 1 | Layer 2 | in PBS | in Methanol | Site/% |
| A | 1% Polymer, | 1% Polymer | 0.077 | 0.020 | 77.8 |
| | 1% Drug | | | | |
| B | 2% Polymer, | 2% Polymer | 0.059 | 0.017 | 77.6 |
| | 1% Drug | | | | |
| C | 1% Polymer | 1% Polymer, | 0.014 | 0.027 | 60.29 |
| | | 1% Drug | | | |
| D | 2% Polymer | 2% Polymer, | 0.039 | 0.019 | 67.24 |
| | | 1% Drug | | | |

It was seen that some flaking was observed with the 2% polymer coated balloons, and therefore 1% polymer coated balloon samples were the more durable samples. With observations made with a microscope, sample C showed the most promising durability overall.

The UV-VIS data showed a slower drug release for the samples in which the drug was in the outside layer.

### Example 7

In this example, 5 balloons were dip coated with a methanol solution containing 20% Pluronic F108 and 8% dipyridamole.

Each coated balloon was then expanded into methanol for 5 minutes to fully strip the balloon of its coating. These solutions were then analysed by UV-VIS to obtain the drug concentration and drug dose. The results are displayed in Table 5.

TABLE 5

| | Absorbance | Drug Amount/μg | Drug Dose/μgmm⁻² |
|---|---|---|---|
| 20% Polymer & | 1.271 | 638.43 | 1.47 |
| 8% Dipyridamole | 1.190 | 597.42 | 1.38 |
| | 1.167 | 585.77 | 1.35 |
| | 1.273 | 639.44 | 1.48 |
| | 1.268 | 606.53 | 1.38 |

These evaluations showed that using 20% polymer and 8% dipyridamole resulted in a drug dose of 1.40±0.10 μmm⁻² being loaded onto the surface of the balloon.

### Example 8

The same method carried out in Example 7 was used, except a methanol solution containing 20% Pluronic F108 and 8% sirolimus was used for dip coating. The results displayed in Table 6 were obtained.

TABLE 6

| | Absorbance | Drug Amount/μg | Drug Dose/μgmm⁻² |
|---|---|---|---|
| 20% Polymer & | 1.412 | 435.30 | 1.00 |
| 8% Sirolimus | 1.373 | 423.28 | 0.976 |
| | 1.529 | 471.37 | 1.09 |
| | 1.631 | 502.81 | 1.16 |

These evaluations showed that using 20% polymer and 8% sirolimus resulted in a drug dose of 1.06±0.10 μgmm⁻² being loaded onto the surface of the balloon.

### Example 9

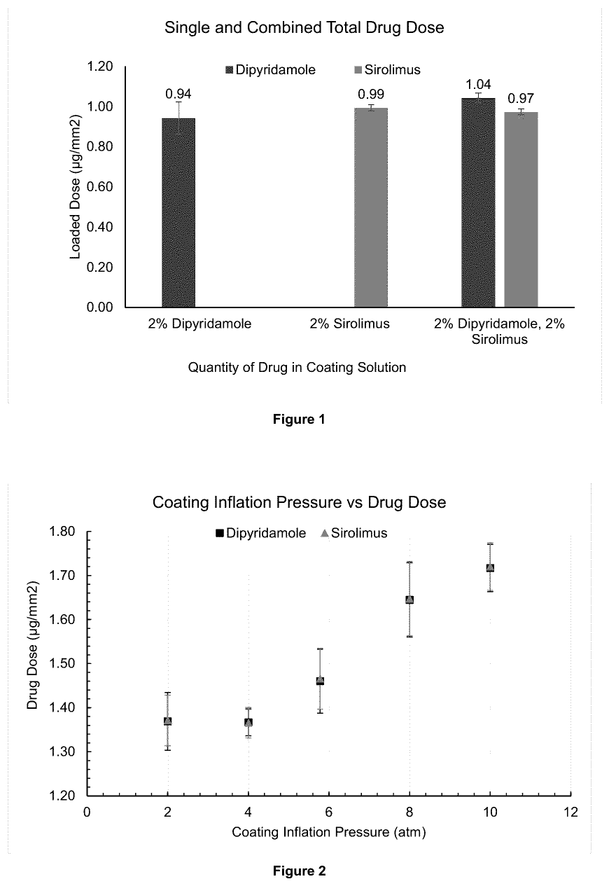

In this example, balloons were coated with a methanol solution containing 1% Pluronic F108 and 2% of either dipyridamole, sirolimus, or dipyridamole and sirolimus. After drying, each balloon had its coating stripped in methanol, which was analysed via UV-VIS to measure the quantity of each drug loaded onto the balloon. This is displayed in FIG. 1.

It was observed that all three coating solutions enabled similar quantities of the respective drug/drugs to be adhered to the balloon surface. The loaded dose of each drug in all cases was between 0.94-1.04 μg/mm².

### Example 10

In this example, 6 balloons were dip coated with a 0.3% dipyridamole methanol solution. The solution also contained Pluronic F108 in the quantities shown below in Table 7.

Each balloon was eluted into a vial of methanol to fully strip the balloons of their coating. Each solution was then analysed by UV-VIS to calculate the drug concentration and thus the loaded drug dose. These are displayed in Table 7.

TABLE 7

| F108 Percentage | UV-VIS | Calculated Drug | Calculated Drug |
|---|---|---|---|
| by Weight/% | Absorbance | Amount/μg | Dose/μgmm⁻² |
| 50 | 0.183 | 87.54 | 0.202 |
| 50 | 0.182 | 87.04 | 0.201 |
| 20 | 0.053 | 21.72 | 0.051 |
| 20 | 0.061 | 25.77 | 0.059 |
| 5 | 0.020 | 5.01 | 0.012 |
| 5 | 0.022 | 6.03 | 0.014 |

It is observed that the greater concentration of polymer used in the coating solution, the more coating was adhered to the surface of the balloon, resulting in a high drug dose.

### Example 11

In this example, 5 balloons were dip coated at room temperature with a methanol solution containing 20% Pluronic F108, 8% dipyridamole and 8% sirolimus by mass. These samples were coated at various balloon inflation pressures; 2 atm, 4 atm, 6 atm, 8 atm (nominal) and 10 atm. The balloons were then left to fully elute into methanol, which was analysed by UV-VIS. The results are displayed in FIG. 2.

FIG. 2 shows the relation between inflation pressure and loaded drug dose. Coating at a lower pressure means that less surface area of the balloon is exposed to the drug solution, and therefore less coating is adhered to the surface of the balloon, resulting in a lower drug dose on the surface of the balloon.

### Example 12

In this example, balloons were dip coated in a methanol solution containing 20% Pluronic F108, 8% dipyridamole, and 8% sirolimus. Half of the coated balloons were sterilised to assess the effect of sterilisation on the total loaded drug content.

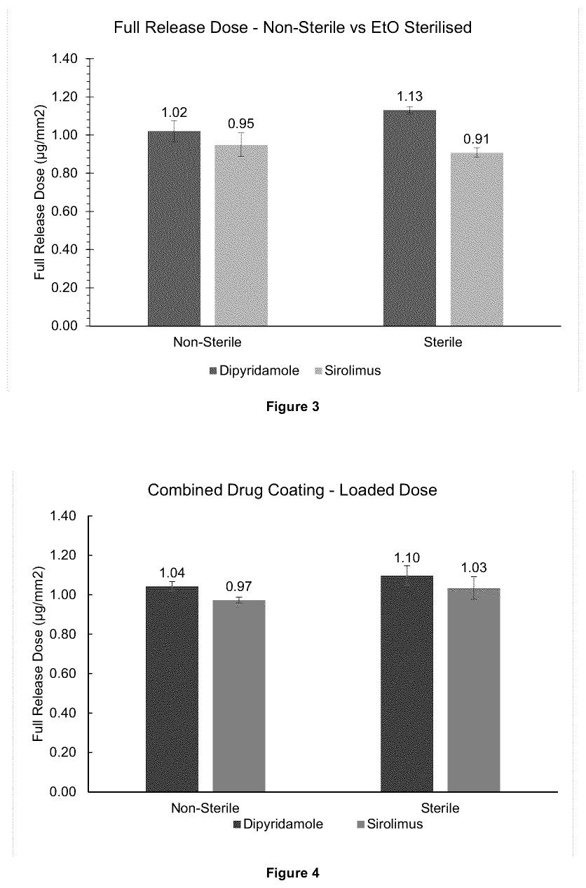

The total drug content of both the sterilised and non-sterilised balloons was assessed by releasing each balloon in methanol, which was then analysed by UV-VIS spectroscopy to calculate the dose per unit area on the balloon, as shown in FIG. 3. The ratios of dipyridamole to sirolimus for non-sterile, and sterilised balloons were 1.07 and 1.24 respectively.

The ratios are both generally close to unity, indicating minimal preferential binding of either drug to the balloon surface and polymer excipient.

### Example 13

In this example, balloons were spray coated with a methanol solution containing 1% Pluronic F108, 2% dipyridamole, and 2% sirolimus. Half of the coated balloons were sterilised to assess the effect of sterilisation on the total loaded drug content, and the ratio of loaded dipyridamole to sirolimus.

The total drug content of both the sterilised and non-sterilised balloons was assessed by releasing each balloon in methanol, which was analysed by UV-VIS to calculate the dose per unit area on the balloon. The results are displayed in FIG. 4.

The ratios of dipyridamole to sirolimus for non-sterile, and sterilised balloons were 1.07 and 1.06 respectively. These are both close to unity, indicating minimal preferential binding of either drug to the balloon surface and polymer excipient.

The detected quantity of loaded drug did not change after sterilisation, indicating that the coating is resilient enough to withstand the effects of sterilisation.

### Example 14

In this example, a hydrogel was used to mimic the arterial tissue wall to assess the drug release profile and simulate the tracking and deployment process.

Based on the process described in ASTM F3320-18, folded balloons were pushed through a 37° C. heated anatomical model filled with PBS for 30 seconds. At the end of the track, balloons were then inflated to nominal pressure inside the hydrogel for 1 minute. The PBS inside the track was collected and replaced, and the balloon withdrawn back through the track. The PBS in the track was collected, and the coated balloon was inflated to nominal pressure for a final drug release in methanol for 5 minutes. The drug concentration in the two PBS samples and methanol sample was measured by UV-VIS. This was used to calculate the drug dose delivered to the hydrogel site.

In this example, the drug release profiles of balloons dip coated with methanol solutions containing 20% excipient Pluronic F108 and 8% of either dipyridamole or sirolimus were assessed. After coating, the balloons were dried and folded.

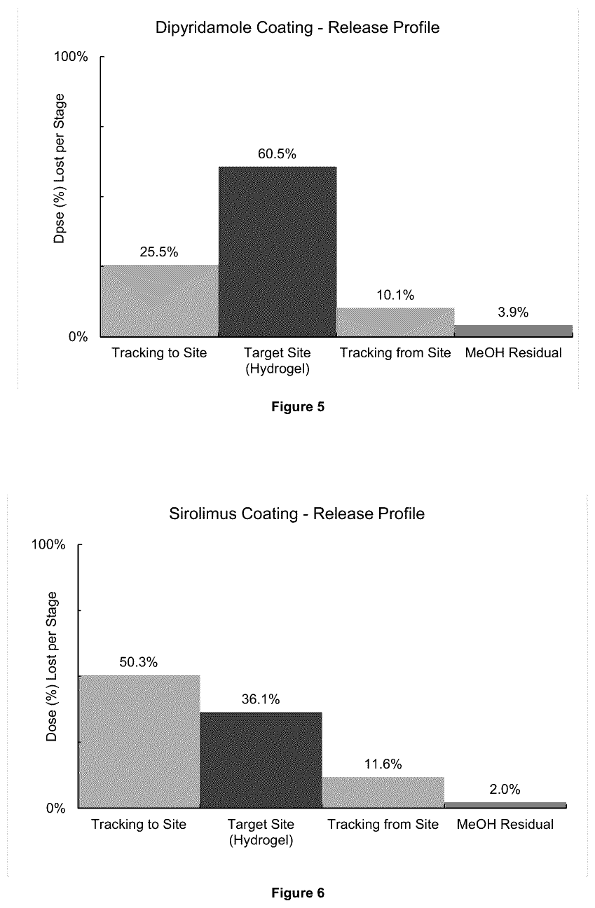

The drug release profiles for the two drug coatings are displayed in FIG. 5 and FIG. 6. When tracking to the site, it was observed that considerably less dipyridamole than sirolimus was lost, hence a greater percentage of the total loaded dose was delivered to the hydrogel. The percentage of the total dose delivered to the hydrogel was 60.5% and 36.1% for dipyridamole and sirolimus respectively.

After deployment in the hydrogel, the release profiles for both drug coatings were similar.

### Example 15

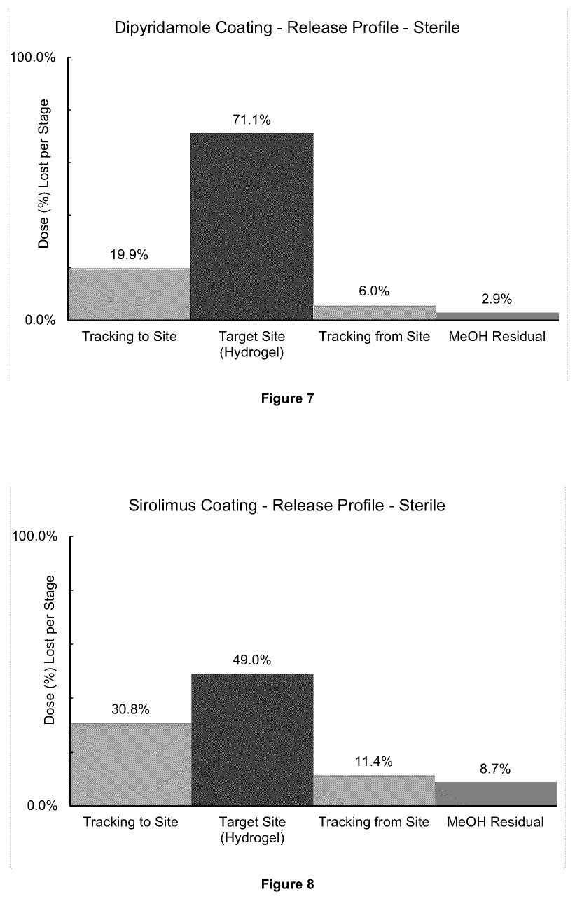

In this example, balloons were spray coated with a methanol solution containing 1% excipient Pluronic F108, and 2% of either dipyridamole or sirolimus. After drying and folding the balloons were sterilised. The balloons were put through a simulated anatomical model tracking and deployment process as described in ASTM F3320-18 and Example 14. The percentage of the total loaded drug dose lost during each stage of the simulated tracking process was measured via UV-VIS and is plotted in FIG. 7 and FIG. 8.

In both cases, the most of the loaded drug (71.1% for dipyridamole, 49.0% for sirolimus) was released during deployment to the hydrogel target site.

### Example 16

In this example, the effect of sterilisation on simulated tracking results was assessed. Balloons were dip coated with a methanol solution containing 20% Pluronic F108, 8% dipyridamole, and 8% sirolimus. The balloons were then dried and folded. Half the balloons were sent for sterilisation and half were kept for a non-sterile control.

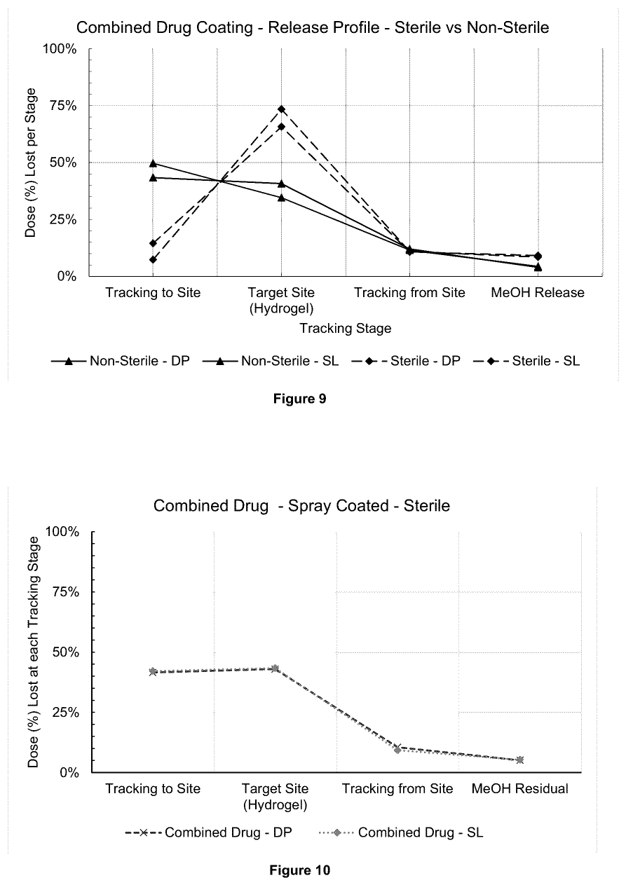

Following the method described in ASTM F3320-18 and Example 14, simulated tracking and deployment was performed using a benchtop tracking fixture consisting of an anatomical model path wrapped in a 37° C. heated sheath, and a hydrogel mock-vessel. The results are displayed in FIG. 9.

It was found that EtO sterilisation vastly increased the percentage of the loaded drug delivered to the mock vessel from =35% to =70%. The quantity of drug lost in transit to the hydrogel site was reduced from =50% to <15% in the sterilised samples. After deployment in the gel, all balloons gave similar release profiles, indicating that most of the drug coating is released after deployment in both sterile and non-sterile samples.

### Example 17

In this example, balloons were spray coated in a methanol solution containing 1% Pluronic F108, 2% dipyridamole, and 2% sirolimus. After drying and folding, the balloons were sterilised.

Following the method described in ASTM F3320-18 and Example 14, simulated tracking and deployment was performed using a benchtop tracking fixture.

The results displayed in FIG. 10 demonstrate nearly identical behaviour of the two drugs in the simulated tracking fixture. In this example, the largest percentage of drug release occurred at the hydrogel target site (43.0-43.3%)

1. A coating composition for application to an expandable medical device comprising
a polymeric compound comprising a central portion and two end portions wherein either the central portion is hydrophobic and the end portions are hydrophilic or the central portion is hydrophilic and the end portions are hydrophobic;
at least one active agent wherein the active agent is hydrophobic when the central portion of the polymeric compound is hydrophobic and the active agent is hydrophilic when the central portion of the polymeric compound is hydrophilic; and
a solvent;
wherein in use the at least one active agent is encapsulated by the polymeric compound; and wherein at least one active agent is a vasodilator and/or an anti-platelet agent.

2. A coating composition for application to an expandable medical device comprising
a polymeric compound comprising a central portion and two end portions wherein either the central portion is hydrophobic and the end portions are hydrophilic or the central portion is hydrophilic and the end portions are hydrophobic;
a plurality of active agents wherein at least one active agent is hydrophobic when the central portion of the polymeric compound is hydrophobic and at least one active agent is hydrophilic when the central portion of the polymeric compound is hydrophilic; and
a solvent;
wherein in use the at least one active agent is encapsulated by the polymeric compound.

3. The coating composition of claim 1, wherein an active agent is dipyridamole.

4. The coating composition of claim 1, wherein an active agent is sirolimus.

5. The coating composition of claim 1, wherein the active agents comprise an anti-proliferative agent and a vasodilator and/or anti-platelet agent.

6. The coating composition of claim 5 wherein the anti-proliferative agent is sirolimus and the vasodilator and/or anti-platelet agent is dipyridamole.

7. The coating composition of claim 1, wherein at least one active agent is hydrophilic and/or hydrophobic.

8. The coating composition of claim 1, wherein the polymeric compound is a triblock copolymer.

9. The coating composition of claim 1, wherein the Mₙ of the polymeric compound is from 13,000 to 50,000 Da.

10. The coating composition of claim 1, wherein the triblock copolymer is a poloxamer derivative.

11. The coating composition of claim 1, wherein the coating composition is a dip coating composition; and wherein each active agent is present in the organic solvent from 1% to 30% by weight.

12. The coating composition of claim 1, wherein the coating composition is a spray coating composition; and wherein each active agent is present in the organic solvent from 1% to 5% by weight.

13. The coating composition of claim 1, wherein the polymeric compound is present in the solvent at less than 50% by weight.

14. The coating composition of claim 1, wherein the coating composition is a dip coating composition; and wherein the polymeric compound is present from 10% to 30% by weight by weight.

15. The coating composition of claim 1, wherein the coating composition is a spray coating composition; and wherein the polymeric compound is present from 0.5% to 5% by weight.

16. A coating composition system for application to an expandable medical device, the coating system comprising:
a) a first coating composition comprising a first polymeric compound comprising a central portion and two end portions wherein either the central portion is hydrophobic and the end portions are hydrophilic or the central portion is hydrophilic and the end portions are hydrophobic; a first active agent wherein the first active agent is hydrophobic when the central portion of the first polymeric compound is hydrophobic and the first active agent is hydrophilic when the central portion of the first polymeric compound is hydrophilic; and a first solvent; wherein in use the first active agent is encapsulated by the first polymeric compound; and wherein the first active agent is a vasodilator and/or an anti-platelet agent; and
b) a second coating composition comprising a second polymeric compound comprising a central portion and two end portions wherein either the central portion is hydrophobic and the end portions are hydrophilic or the central portion is hydrophilic and the end portions are hydrophobic; a second active agent wherein the second active agent is hydrophobic when the central portion of the second polymeric compound is hydrophobic and the second active agent is hydrophilic when the central portion of the second polymeric compound is hydrophilic; and a second solvent; wherein in use the second active agent is encapsulated by the second polymeric compound;
and wherein the first and second active agents are different.

17. The coating composition system of claim 16 wherein the first and second active agents are independently hydrophilic and/or hydrophobic, for example wherein the first active agent is hydrophilic and the second active agent is hydrophobic.

18. The coating composition system of claim 17, wherein the first active agent is dipyridamole.

19. The coating composition system of claim 16, wherein the second active agent is sirolimus.

20. The coating composition system of claim 16, wherein the first and second polymeric compound are each a triblock copolymer.

21. The coating composition system of claim 16, wherein the Mₙ of each of the first and second polymeric compound is from 13,000 to 50,000 Da.

22. The coating composition system of claim 20, wherein the triblock copolymer is a poloxamer derivative.

23. The coating composition system of claim 16, wherein each of the first and second coating composition is a dip coating composition; and wherein each active agent is present in the organic solvent from 1% to 30% by weight.

24. The coating composition system of claim 16, wherein each of the first and second coating composition is a spray coating composition; and wherein each active agent is present in the organic solvent from 1% to 5% by weight.

25. The coating composition system of claim 16, wherein the polymeric compound is present in the solvent at less than 50% by weight.

26. The coating composition system of claim 16, wherein each of the first and second coating composition is a dip coating composition; and wherein the polymeric compound is present from 10% to 30% by weight by weight.

27. The coating composition of claim 16, wherein each of the first and second coating composition is a spray coating composition; and wherein the polymeric compound is present from 0.5% to 5% by weight.

28. An expandable medical device coated on at least a portion of the surface thereof with a coating, the coating being derived from a coating composition according to claim 1, and/or a coating system, the coating system being derived from a coating composition system according to claim 16.

29. A method of coating an expandable medical device comprising the steps of
(i) providing a coating composition according to claim 1 and/or a coating composition system according to claim 16;
(ii) applying the coating composition and/or coating composition system of step (i) to at least a portion of the surface of the expandable medical device; and optionally
(iii) applying a mixture of a polymeric compound comprising a central portion and two end portions wherein either the central portion is hydrophobic and the end portions are hydrophilic or the central portion is hydrophilic and the end portions are hydrophobic and optionally a solvent to at least a portion of the surface of the expandable medical device coated in step (ii).

30. The method according to claim 29 wherein the coating composition and/or coating composition system is applied by dip coating or spray coating.

31. The method of delivering at least one active agent to a target site in a lumen of a human or animal body for treating a condition associated with said lumen comprising: locating an expandable medical device according to claim 28 at the target site; and expanding the expandable medical device such that the surface of the device is in contact with the target site.

32. The coating composition of claim 1, coating composition system of claim 16, expandable medical device of claim 28 or the method of claim 29 wherein the device is a balloon catheter.

33. The coating composition, coating composition system, expandable medical device or method of claim 32 wherein the balloon catheter has a hydrophobic or hydrophilic surface.

34. The coating composition, coating composition system, expandable medical device or method of claim 33, wherein the balloon catheter has a hydrophobic surface.
